(19) United States
(12) Patent Application Publication
(10) Pub. No.: US 2023/0221276 A1
(43) Pub. Date: Jul. 13, 2023

(54) FREE ACTIVE CHLORINE MEASUREMENT SYSTEM AND METHOD

(71) Applicant: Spraying Systems Co., Wheaton, IL (US)

(72) Inventors: John Tyler Williams, St. Charles, IL (US); Nicholas Williams, Aurora, IL (US); Jack Bernard, Oak Park, IL (US)

(21) Appl. No.: 18/095,173

(22) Filed: Jan. 10, 2023

Related U.S. Application Data
(60) Provisional application No. 63/298,009, filed on Jan. 10, 2022.

(51) Int. Cl. G01N 27/27; G01N 27/416

(52) U.S. Cl. CPC G01N 27/27; G01N 27/4167; G01N 27/4168

(57) ABSTRACT

A system and method for measuring concentration of oxidation ions in a solution includes a conduit that contains the solution at least temporarily, a pH sensor associated with the conduit and configured to provide a pH signal indicative of a pH of the solution, an oxidation reduction probe (ORP) associated with the conduit and configured to provide an ORP signal indicative of an oxidation reduction of the solution, and a controller configured to receive and process the pH signal and the ORP signal, wherein the controller calculates a concentration of oxidation ions in the solution based on the pH and ORP signals.

### CROSS-REFERENCE TO RELATED APPLICATIONS

The present patent application claims priority to U.S. Provisional Patent Application No. 63/298,009, filed on Jan. 10, 2022, which is incorporated herein in its entirety by reference.

### FIELD OF THE DISCLOSURE

The present disclosure relates to systems and methods for generating and using sanitizing and disinfecting agents and, more particularly, to systems that utilize free active chlorine (FAC) solutions.

### BACKGROUND OF THE DISCLOSURE

One of the most used sanitizing and disinfecting agents is oxidative chlorine species known as Free Available Chlorine (FAC). The ability to measure the Free Available Chlorine (FAC) concentration in these sanitizing/disinfection solutions is critical to ensure solution efficacy, and prove it falls within required parameters. As much of the industry is aware, most FAC working concentrations fall within very low levels, 2-10 ppm of FAC. Analytical methods and meters or sensors have been developed to measure these lower levels. However, there is an inability to economically measure higher concentrations (50+ ppm range), especially inline. As higher concentrations become available and the new technologies such as on-site generation (OSG) become more common place, inline chlorine measurements become desirable. Existing sensors are cost prohibitive (particularly for high-FAC concentration range units), and/or require reagents and controlled sample sizes to be used, which requires regular maintenance and/or user intervention. These current techniques are often not easily applied to emerging technologies outside of high-end industrial applications.

### BRIEF SUMMARY OF THE DISCLOSURE

The present disclosure describes systems and methods using algorithms for free active Chlorine (FAC) measurement utilizing low-cost sensors. Advantageously, the sensors utilized do not affect or interact with the solution whose concentration of FAC is being measured, and can be used on a continuous basis as part of a process and/or on a permanent basis in containers storing such solutions to ensure that a proper concentration remains present while a product is stored at a facility or on the shelf. To measure inline or static FAC inexpensively, reliably, and with acceptable accuracy (within 5%), and without use of reagents, the proposed invention utilizes an algorithm that utilizes data measured by an Oxidation Reduction Potential (ORP) sensor, pH sensor, and known temperature to calculate an FAC reading that can be provided inline and/or statically within a storage tank to provide an indication of tank solution health and suitability for use.

### BRIEF DESCRIPTION OF THE SEVERAL VIEWS OF THE DRAWINGS

FIG. 1 is a diagram of a system in accordance with the disclosure.

FIG. 2 is a section view of an inline sensor arrangement in accordance with the disclosure.

FIG. 3 is a section view of an alternative inline sensor arrangement in accordance with the disclosure.

FIG. 4 is a section view through a portion of a holding tank having a sensor arrangement in accordance with the disclosure.

### DESCRIPTION OF PREFERRED EMBODIMENTS

Exemplary embodiments of the present disclosure include a system and method that determining, calculating or otherwise measuring FAC concentration in a solution having a high concentration, or more than 50 ppm, for example, in the neighborhood of 500 ppm, 1000 ppm, or more of FAC. The systems and methods are configured to measure FAC concentration either inline, for example, when on onsite production is used, and/or continuously while the solution is stored for later use. In one embodiment, the measurement system includes use of a pH sensor in concert with an oxidation reduction probe (ORP). In an alternative embodiment, a temperature probe is also used. The sensor(s) and probe provide information to a controller, which can be physical or virtual, which processes the information to determine or calculate the FAC concentration in a stream and/or holding tank.

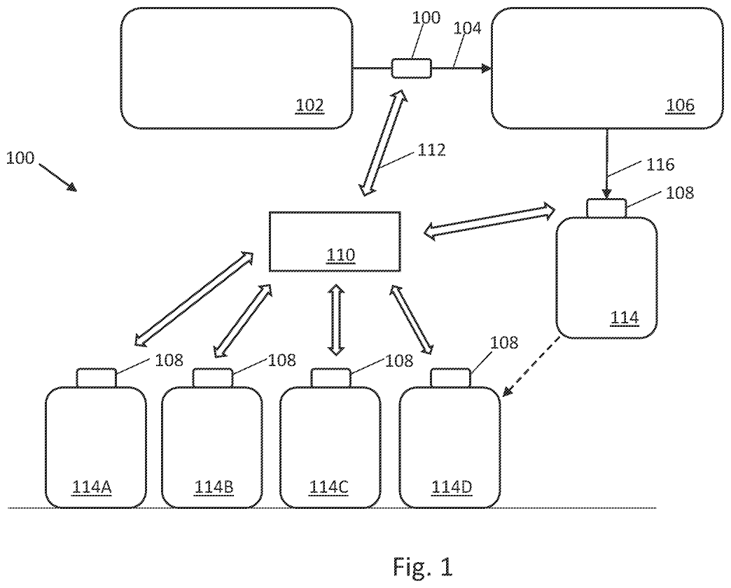

A system 100 for production and storage of an FAC solution is shown in FIG. 1. The system 100 includes a production plant 102 that operates to produce a solution having a high concentration of FAC. Any suitable production plant can be used. For example, the production plant 102 may be an electrolyzing system using brine such as the system described in U.S. Pat. No. 10,577,262, which granted on Mar. 3, 2020, to Cronce et. al. and is incorporated herein in its entirety by reference.

In the system 100, solution containing high concentration of FAC, for example, a concentration of 50. 500, 1000 ppm or more, is provided at an outlet conduit 104 to a facility 106 for use, for example, as a disinfectant, to treat a water supply, and the like. A sensor pack 108 is disposed inline along the conduit 104 to measure the concentration of FAC in the solution in real time during operation. The sensor pack 108, which is described and shown in further detail in the figures that follow, includes a pH sensor, an oxidation reduction probe (ORP) and, optionally, a temperature sensor. The sensor pack components are communicatively connected to a controller 110 via a wired or wireless connection 112. As can be appreciated, the controller 110 may be integrated with the sensor pack as a local controller or the controller 110 may alternatively be remote to the sensor pack either onsite or virtually in a cloud and use appropriate communication protocols to receive signals from the sensor pack 108 that are indicative of the respective values measured by the various sensors (pH, ORP and Temperature).

In addition, or instead of the facility 106, excess solution may be stored in a holding tank 114. In the illustrated embodiment, the tank 114 includes a sensor pack 108 at its inlet and along a conduit 116 that provides FAC containing solution to the tank 114. The tank 114 may be one of many tanks 114 that are filled and then stored at a facility for later use. In the illustration of FIG. 1, a tank 114 is shown while being filled, and previously filled tanks 114A, 114B, 114C, and 114D are stored for later use. Each of the tanks 114 contains a respective sensor pack 108 that is communicatively linked with the controller 110. Advantageously, the sensor pack 108 of the tank 114 can monitor the FAC concentration of the solution filling the tank 114 to ensure quality of the solution for later use. Information on the quality of the solution filling the tank 114 can be logged and provided with the tank to an end user. Further, the sensor packs 108 of the tanks 114A-114D can continuously monitor the quality of the solution stored in the tanks to ensure consistent quality and preservation of the FAC content of the stored solution prior to and during use later by the end user.

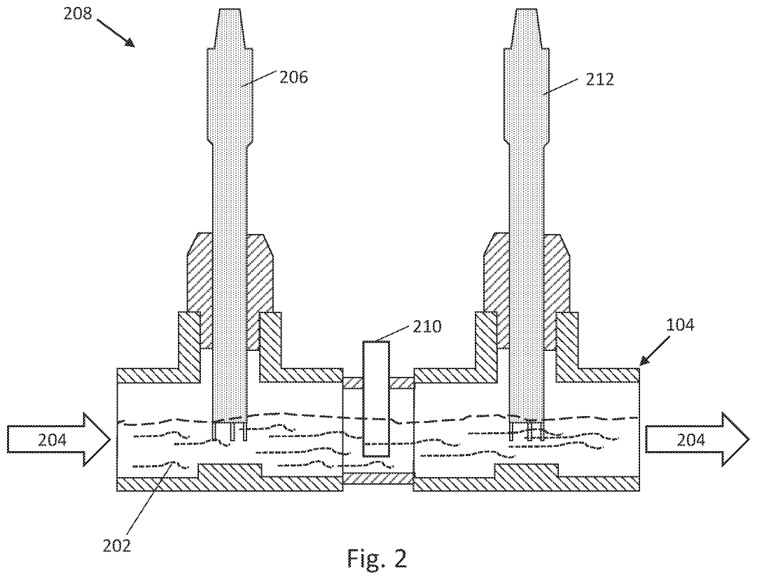

A cross section view through a first embodiment of a sensor pack 108 is shown in FIG. 2. In this embodiment, a sensor pack 208 is shown disposed along a fluid conduit, for example, the fluid conduit 104 (FIG. 1) that carries the FAC containing solution from the production plant 102. As can be seen here, solution 202 flows in the conduit 104 in the direction of arrows 204 (from left to right, in the orientation shown). The solution 202 has sufficient flow rate to fill at least half the conduit or at least fill the conduit at a sufficient height to allow the solution to touch the sensing ends of sensors disposed on the conduit.

As the solution travels through the conduit 104 it passes first over a sensing end of a pH sensor 206, then over a temperature sensor or thermocouple 210, and then over the sensing end of an oxidation reduction probe (ORP) 212. Suitable examples of sensors include but are not limited to AtalsScientific™ Lab grade ORP Probe (ENV-40-ORP), Milwaukee Instruments Double Junction pH electrode (MA911B/2) and Omega compact RTD Temperature Sensor (RTDM12-1/8NPT-3MM-13MM-A).

To increase result accuracy, the sensors 206, 210 and 212 can be disposed as close together as possible along the flow of solution through the conduit to ensure that they are all measuring as close to the same volume of solution as possible. Of course, if the solution has generally constant FAC concentration or is mixed upstream of the sensor pack 208, the close positioning of the sensors may not affect the accuracy of the measurements. The sensors of the sensor pack 208 are communicatively connected to a controller (such as the controller 110 shown in FIG. 1) that receives and processes their signals to estimate or calculate the FAC concentration in the solution 202. In one embodiment, the controller converts the signals from the probes to pH and mV values, which are then used in a calculation using the Nernst equation (Equation 1a below) to solve for the membrane potential at equilibrium within the production plant.

The reduction/oxidation relationship Q (Equation 1b) is obtained by using the input pH (reduction) of the production plant and the output pH (oxidation) of the production plant. The calculated value from Equation 1a is then compared to the measured potential from the ORP probe, yielding a AORP value (Equation 2a). The accuracy can be further improved by adjusting the AORP with an experimental efficiency factor (Equations 3a-d), which then outputs an FAC value. These equations are shown below:

$$( Nernst Equation ) : E = {E}^{0} - \frac{R T}{z F} ln ( Q ) \qquad Equation la$$

$$E = {ORP}_{calc}$$

$$( \frac{red}{o x} ) : Q = \frac{1 {0}^{- ( 14 - φ )}}{1 {0}^{- ω}} \qquad Equation 1 b$$

$$\begin{matrix}Input pH : φ \\ Output pH : ω\end{matrix}$$

$$Δ ORP = {ORP}_{m e a s} - {ORP}_{calc} \qquad Equation 2 a$$

$${α}_{O R P} = \frac{O R {P}_{c a l c}}{O R {P}_{m e a s}} ; Unitless accuracy factor \qquad Equation 3 a$$

$${β}_{ORP} = \frac{Δ O R P}{F A {C}_{m e a s}} ; \qquad Equation 3 b$$

$$FA {C}_{meas} measured using high - range FAC photometer$$

$${γ}_{ORP} = \frac{{α}_{O R P}}{{β}_{ORP}} ; Efficiency Factor \qquad Equation 3 c$$

$${FAC}_{calc} = {γ}_{ORP} \cdot Δ ORP ; Final FAC Output \qquad Equation 3 d$$

$${FAC}_{calc} = ( {ORP}_{m e a s} - ( 0.815 - \frac{R * t}{z * F} * ln ( \frac{1 {0}^{- ( 14 - φ )}}{1 {0}^{- ω}} ) ) * 1000 ) * ( 0.815 - \frac{R * t}{z * F} * ln ( \frac{1 {0}^{- ( 14 - φ )}}{1 {0}^{- ω}} ) ) * \frac{1000}{O R {P}_{meas}} \qquad Equation 4$$

During operation, the controller 110 samples continuously or intermittently the solution 202 using the sensors 206, 210 and 212, and applies the sensed values to the equations above to calculate or otherwise determine the FAC concentration of the solution 202. It is contemplated that the “flow” illustrated is a flow that is provided at an output of an industrial process for creating FAC in a solution stream and/or a flow that is collected in a container or reservoir for later use or dilution for use in a disinfecting application. In this embodiment the pH, ORP measurement, a constant temperature, and all the remaining parameters that may be needed are provided to a controller for calculating the FAC concentration in the flow.

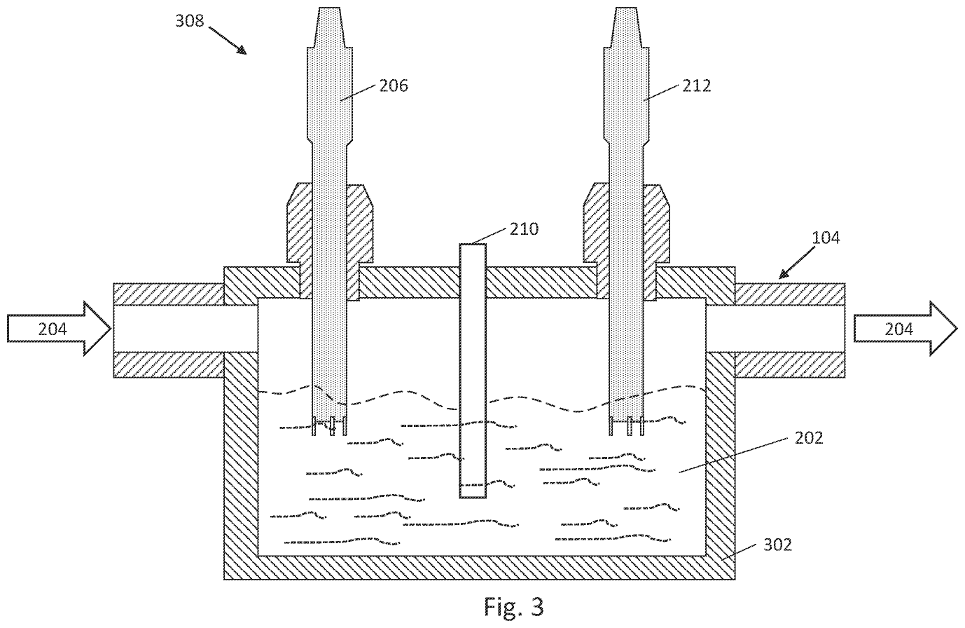

An alternative embodiment for a sensor pack 308 is shown in FIG. 3. In this embodiment, a catch basin 302 is used to collect solution 202 in applications where the production flow rate of solution 202 may be insufficient to fill the conduits 104 to a sufficient height to provide a reliable reading at the sensors 206, 210 and 212. Alternatively, the catch basin 302 can also be used even in application where a sufficient flow of solution is present, in which case the volume of solution 202 collecting in the catch basin results in localized mixing of the solution to normalize the FAC concentration for purpose of measurement. In FIG. 3, the same reference numerals are used to denote the same or similar corresponding components as those described and shown relative to the embodiment of FIG. 2 for simplicity. In the embodiment of FIG. 3, all meters are disposed within the same receptacle, which improves sensor durability and increases increase accuracy of measurements by ensuring that the probes remain submerged continuously during operation.

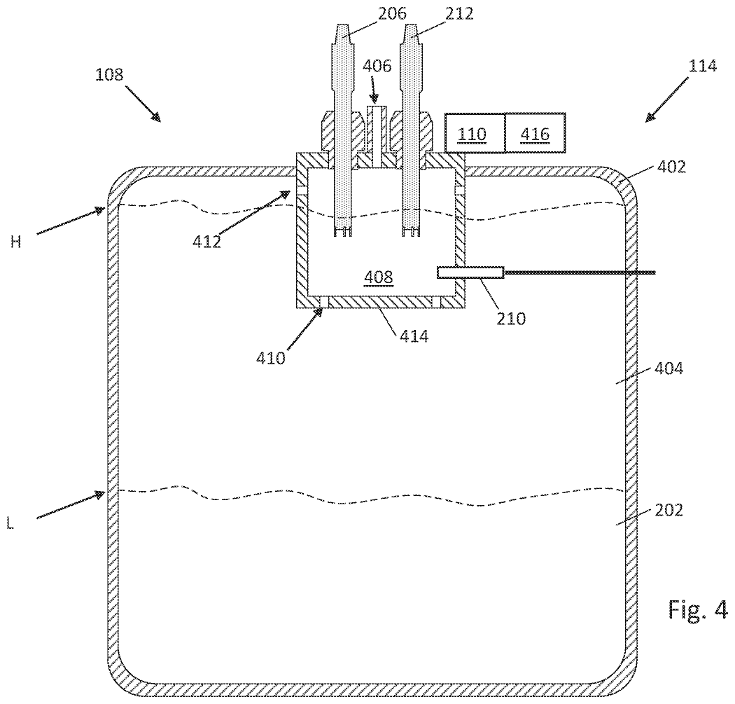

A cross section through a holding tank 114 (FIG. 1) is shown in FIG. 4. In this embodiment, the tank 114 includes a shell 402 that encloses a space 404 that is filled with solution 202. The shell 402 includes a fill opening 406 which permits filling of the tank 114 with solution. An outlet opening to empty the tank 114 is not shown for simplicity. The fill opening 406 deposits an incoming flow of solution 202 into a pre-chamber 408 that has one or more controlled area drain openings 410 and one or more overflow openings 412, all of which are open to the internal space 404 of the shell 402. The pre-chamber 408 is formed within a sampling cup 414 that is formed internal to the shell 402 and occupies a portion of the space 404 of the tank 114.

During a filling process, incoming solution 202 first fills the sampling cup 414 and thus exposes the sensors 206, 212 and 210 to the solution for purpose of measurement. In other words, the sampling cup 414 and the sensors 206, 210 and 212 form part of the sensor pack 108 associated with the tank 114. While the tank is filling, the level of solution 202 within the tank 114 is low, for example, below the drain openings 410, at a level, L. As the tank 114 is filled to capacity, the level of solution 202 in the tank 114 reaches a high level, H, which submerges the sampling cup 414 and also covers at least the drain openings 410. In this condition, the tank may be removed and stored for later use of the solution 202 contained therein. Advantageously, the sensors 206, 210 and 212 remain submerged in the solution 202 contained within the tank and are used to continuously monitor the FAC concentration of the solution 202 stored within the tank 114, even while the tank is at a storage area and not fluidly connected to a solution supply or return.

In the embodiment shown in FIG. 4, the probes are inserted into a sample cup which has a drain hole in the bottom and an overflow near the top, and which sits within the solution storage tank directly beneath the inlet to the tank. This allows for the real-time measurement of the solution being produced, as well as a measurement of the entire tank once it is full, which is especially important when specific chlorinated species have a shelf life of less than 30 days.

The systems and methods described herein are useful in that they require a small fraction of the cost of other inline chlorine measurement devices, can measure high FAC levels in real-time, are simple to calibrate and can be applied to very high range (1000+ ppm) FAC applications.

The methods described herein have been confirmed to be accurate using laboratory measurements. For example, 250+ sample solutions were tested, and their concentrations were determined using existing photometric methods. These results were then compared to the calculated concentrations acquired by the inventive system and were found to be within 5% of the actual value.

In addition to FAC measurements, the systems and methods described herein are useful for measuring any oxidation ion in a solution. In principle, it is believed that the systems and methods in accordance with the disclosure account for electrons—by comparing the free electrons in an input and in an output of a process to determine a difference. The difference represents the amount of ion change induced by the process.

In the embodiment of FIG. 4, depending on how the draining orifice from the cup is selected, the sensors can always remain wetted to measure not only the FAC in the input stream, but also the FAC concentration of the stored solution to determine the solution efficacy during storage and to enable adjustments to the dilution of the solution for use. Current FAC methods destroy the sample—the sensors in accordance with the disclosure don't affect the solution at all and can remain in contact with the solution, if desired to monitor the concentration of the FACs on a shelved product, indefinitely.

The embodiment can use either a known temperature based on the application, or a user-input temperature. The temperature can be set to a single value near the actual liquid temperature because small differences in temperature have a negligible effect on the calculated FAC, with experimental temperature changes of 5° C. yielding less than 0.5% change in the output.

In one embodiment, the sensor pack 108 may include the controller integrated therewith and also a display 416 for displaying in real time the concentration of the parameters measured. In addition to displaying these values on the device itself, the embodiment has the ability to upload the data stream of pH and FAC to an external destination via Wi-Fi/cellular connectivity, allowing for remote monitoring of installations.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and “at least one” and similar referents in the context of describing the invention (especially in the context of the following claims) are to be construed to cover both the singular and the plural, unless otherwise indicated herein or clearly contradicted by context. The use of the term “at least one” followed by a list of one or more items (for example, “at least one of A and B”) is to be construed to mean one item selected from the listed items (A or B) or any combination of two or more of the listed items (A and B), unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

What is claimed is:

1. A system for measuring concentration of oxidation ions in a solution, comprising:
a conduit that contains the solution at least temporarily;
a pH sensor associated with the conduit and configured to provide a pH signal indicative of a pH of the solution;
an oxidation reduction probe (ORP) associated with the conduit and configured to provide an ORP signal indicative of an oxidation reduction of the solution; and
a controller configured to receive and process the pH signal and the ORP signal;
wherein the controller calculates a concentration of oxidation ions in the solution based on the pH and ORP signals.

2. The system of claim 1, wherein the oxidation ions are free active chlorine (FAC) in the solution.

3. The system of claim 1, wherein the concentration is more than 50 ppm.

4. The system of claim 1, wherein the concentration is more than 1000 ppm.

5. The system of claim 1, further comprising a temperature sensor associated with the conduit and configured to provide a temperature signal indicative of a temperature of the solution to the controller, and wherein the controller calculates the concentration further based on the temperature of the solution.

6. The system of claim 1, wherein the controller calculates the concentration of oxidation ions in the solution according to the Nernst Equation.

7. The system of claim 1, wherein the conduit includes a catch basin into which solution is collected and with which the pH sensor and the ORP are associated.

8. The system of claim 1, wherein the conduit includes a sampling cup into which solution is collected and with which the pH sensor and the ORP are associated, and wherein the sampling cup is disposed within a storage tank shell and occupies a portion of an internal tank space.

9. A sensor pack for measuring concentration of oxidation ions in a solution, comprising:
a cavity adapted to contain the solution;
a pH sensor associated with the cavity and configured to provide a pH signal indicative of a pH of the solution within the cavity;
an oxidation reduction probe (ORP) associated with the cavity and configured to provide an ORP signal indicative of an oxidation reduction of the solution within the cavity;
a temperature sensor associated with the cavity and configured to provide a temperature signal indicative of a temperature of the solution within the cavity; and
a controller configured to receive and process the pH signal, the ORP signal, and the temperature signal;
wherein the controller calculates a concentration of oxidation ions in the solution within the cavity based on the pH, temperature, and ORP signals.

10. The sensor pack of claim 9, wherein the oxidation ions are free active chlorine (FAC) in the solution.

11. The sensor pack of claim 9, wherein the cavity is a catch basin disposed in line with a conduit through which the solution is adapted to flow.

12. The sensor pack of claim 9, wherein the cavity is a sampling cup into which solution is collected and with which the pH sensor and the ORP are associated, and wherein the sampling cup is disposed within a storage tank shell and occupies a portion of an internal tank space.

13. A method for measuring concentration of oxidation ions in a solution, the method comprising:
providing a conduit through which the solution is adapted to flow;
providing a pH sensor associated with the conduit and configured to provide a pH signal indicative of a pH of the solution;
providing an oxidation reduction probe (ORP) associated with the conduit and configured to provide an ORP signal indicative of an oxidation reduction of the solution; and
calculates a concentration of oxidation ions in the solution based on the pH and ORP signals.

14. The method of claim 13, wherein the oxidation ions are free active chlorine (FAC) in the solution.

15. The method of claim 13, wherein the concentration is more than 50 ppm.

16. The method of claim 13, wherein calculating the concentration includes utilizing a controller that is communicatively associated with the pH sensor and the ORP, the controller executing non-transitory computer executable instructions stored in tangible media.

17. The method of claim 13, further comprising providing a temperature sensor associated with the conduit and configured to provide a temperature signal indicative of a temperature of the solution to the controller, and wherein calculating the concentration is further based on the temperature of the solution.

18. The method of claim 13, wherein calculating the concentration of oxidation ions in the solution includes applying the Nernst Equation.

19. The method of claim 13, further comprising collecting the solution in a catch basin that is part of the conduit and with which the pH sensor and the ORP are associated.

20. The method of claim 13, further comprising collecting the solution at least temporarily in a sampling cup with which the pH sensor and the ORP are associated, wherein the sampling cup is disposed within a storage tank shell and occupies a portion of an internal tank space.
